(19) United States
(12) Patent Application Publication
(10) Pub. No.: US 2005/0037947 A1
(43) Pub. Date: Feb. 17, 2005

(54) INHIBITION OF DRUG BINDING TO SERUM ALBUMIN

(76) Inventors: Alan J. Bitonti, Acton, MA (US); Vito J. Palombella, Needham, MA (US); James M. Stattel, Leominster, MA (US); Robert T. Peters, West Roxbury, MA (US)

Correspondence Address: FINNEGAN, HENDERSON, FARABOW, GARRETT & DUNNER;LLP, 1300 I STREET, NW, WASHINGTON, DC, 20005, US

(21) Appl. No.: 10/841,949

(22) Filed: May 6, 2004

Related U.S. Application Data
(60) Provisional application No. 60/469,603, filed on May 6, 2003.

(51) Int. Cl. A61K 31/00

(52) U.S. Cl. 514/1

(57) ABSTRACT

The invention relates to improved therapeutics for treating diseases or conditions that provide greater bioavailabilty and more predictable dosing. The invention relates to a chimeric protein comprised of a biologically active molecule linked to an Fc fragment of an immunoglobulin, wherein the chimeric protein binds less serum albumin compared to the same biologically active molecule of the chimeric protein not linked to an Fc fragment of an immunoglobulin. The invention also relates to a method of treating a disease or condition said method comprising administering a chimeric protein comprising a biologically active molecule linked to an Fc fragment of an immunoglobulin, wherein the chimeric protein binds less serum albumin compared to the same biologically active molecule of the chimeric protein not linked to an Fc fragment of an immunoglobulin

This application claims priority to U.S. Provisional Application No. 60/469,603 filed May 6, 2003, which is incorporated by reference.

### FIELD OF THE INVENTION

The invention relates generally to the field of pharmacokinetics and pharmacodynamics. More specifically, the invention relates to methods of increasing the bioavailability and serum levels of a therapeutic agent.

BACKGROUND of the Invention

Serum albumin, the most abundant plasma protein in human plasma, has a concentration of 0.6 mM. It contributes 60% on a per weight basis of the total protein content of plasma. Its presence is not limited to plasma, but can be found throughout the body tissue, most notably in the intestines. A molecule of serum albumin consists of a single non-glycosylated polypeptide chain of 585 amino acids with a molecular weight of 66.5 kD. The conformation of the protein is maintained, in part, by a series of intra-chain disulfide bonds (Clerc et al. 1994, J. Chromatography 662:245). Serum albumin is known to be polymorphic (Carter et al. 1994, Adv. Prot. Chem. 45:153) and the complete amino acid sequence of the most prevalent human form has been described (Dugaiczyk et al. 1982, Proc. Nat. Acad. USA 79:71).

Serum albumin has no associated enzymatic activity and is non-immunogenic. It functions as part of the circulatory system in the transport, metabolism, and distribution of exogenous and endogenous ligands (Rahimipour et al. 2001, J. Med. Chem. 44:3645). It also functions in the maintenance of osmolarity and plasma volume. It has a serum half-life of 14-20 days and is cleared from circulation by the liver (T.A. Waldmann, 1977, Albumin Structure, Function and Uses, Pergamon Press, Princeton, N.J.).

Many compounds, particularly biologically active molecules, e.g., therapeutic drugs, bind reversibly to serum albumin. The pharmacokinetics of an administered drug is greatly influenced by its affinity for serum albumin. A high affinity for serum albumin will reduce the overall free concentration of a therapeutic drug and thus reduce its physiological activity. Therapeutic drug binding to serum albumin can therefore require administration of higher doses of the drug to attain a desired physiological outcome. This in turn increases the risk of side effects. Moreover, circulating complexes of drug and serum albumin may provide a reservoir of drug with unpredictable and uncontrolled release that can contribute to the problems of unpredictable dosing and side effects (Frostell-Karlson et al. 2000, J. Med. Chem. 43:1986).

Accordingly, one aspect of the invention provides a chimeric protein comprising a modified biologically active molecule, wherein the modified biologically active molecule has decreased affinity, or no affinity, for serum albumin and thus both greater bioavailabiltity, and more predictable dosing, compared to the unmodified biologically active molecule. An additional aspect of the invention provides a method of treating a subject having a disease or condition with a chimeric protein comprising a modified biologically active molecule, wherein the modified biologically active molecule binds less serum albumin or no serum albumin compared to the unmodified biologically active molecule. In certain embodiments of the invention, the serum albumin will be human serum albumin.

An aspect of the invention provides a chimeric protein comprising a biologically active molecule and at least a portion of an immunoglobulin constant region. The portion of the immunoglobulin may be an Fc fragment, or a portion that binds FcRn.

### SUMMARY OF THE INVENTION

The invention relates to a method of treating a subject having a disease or condition, comprising administering a chimeric protein to said subject such that the disease or condition is treated, wherein said chimeric protein comprises a biologically active molecule having a modification and wherein, said modification comprises linking said biologically active molecule to at least a portion of an immunoglobulin constant region such that said biologically active molecule having the modification binds less serum albumin, or no serum albumin, compared to the same biologically active molecule without said modification. The portion of the immunoglobulin may be an Fc fragment, or a portion that binds FcRn. In certain embodiments of the invention, the serum albumin will be human serum albumin.

The invention relates to a chimeric protein comprising a biologically active molecule having a modification, wherein said modification comprises linking said biologically active molecule to at least a portion of an immunoglobulin constant region, such that said biologically active molecule binds less serum albumin, or no serum albumin, compared to the same biologically active molecule without said modification.

The invention relates to a method of increasing the unbound serum concentration of a biologically active molecule, said method comprising providing a chimeric protein comprising the biologically active molecule, said biologically active molecule having a modification, wherein said modification comprises linking said biologically active molecule to at least a portion of an immunoglobulin constant region such that said biologically active molecule having said modification binds less serum albumin or no serum albumin compared to the same biologically active molecule without said modification, thus increasing the unbound serum concentration of said biologically active molecule.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1 compares human serum albumin binding to T20, to a chimeric protein comprising T20 linked to an Fc fragment of an immunoglobulin.

FIG. 2 compares human serum albumin binding to a VLA4 antagonist, gonadatropin releasing hormone (GnRH), a chimeric protein comprising GnRH linked to an Fc fragment of an immunoglobulin and a chimeric protein comprising a VLA4 antagonist linked to an Fc fragment of an immunoglobulin.

FIG. 3 shows the amino acid sequence encoding T20(A), T21 (B) T1249(C), N_{CCG}gP41(D) and 5 helix(E).

FIG. 4 shows the amino acid (B) and nucleic acid sequence (A) of an Fc fragment of an immunoglobulin.

### DESCRIPTION OF THE EMBODIMENTS

A. Definitions

Affinity tag, as used herein, means a molecule attached to a second molecule of interest, capable of interacting with a specific binding partner for the purpose of isolating or identifying said second molecule of interest.

Analogs of, or proteins or peptides substantially identical to, the chimeric proteins of the invention, as used herein, means that a relevant amino acid sequence of a protein or a peptide is at least 70%, 75%, 80%, 85%, 90%, 95%, 97%, 98%, 99%, or 100% identical to a given sequence. By way of example, such sequences may be variants derived from various species, or they may be derived from the given sequence by truncation, deletion, amino acid substitution or addition. Percent identity between two amino acid sequences is determined by standard alignment algorithms such as, for example, Basic Local Alignment Tool (BLAST) described in Altschul et al. (1990) J. Mol. Biol., 215:403-410, the algorithm of Needleman et al. (1970) J. Mol. Biol., 48:444-453; the algorithm of Meyers et al. (1988) Comput. Appl. Biosci., 4:11-17; or Tatusova et al. (1999) FEMS Microbiol. Lett., 174:247-250, etc. Such algorithms are incorporated into the BLASTN, BLASTP and “BLAST 2 Sequences” programs (see www.ncbi.nlm.nih.gov/BLAST). When utilizing such programs, the default parameters can be used. For example, for nucleotide sequences, the following settings can be used for “BLAST 2 Sequences”: program BLASTN, reward for match 2, penalty for mismatch −2, open gap and extension gap penalties 5 and 2 respectively, gap x_dropoff 50, expect 10, word size 11, filter ON. For amino acid sequences the following settings can be used for “BLAST 2 Sequences”: program BLASTP, matrix BLOSUM62, open gap and extension gap penalties 11 and 1 respectively, gap x_dropoff 50, expect 10, word size 3, filter ON.

Biologically active molecule, as used herein, means a non-immunoglobulin molecule or fragment thereof, capable of treating a disease or condition or localizing or targeting a molecule to a site of a disease or condition in the body by performing a function or an action, or stimulating or responding to a function, an action or a reaction, in a biological context (e.g. in an organism, a cell, or an in vitro model thereof).

Bioavailability, as used herein, means the extent and rate at which a substance is absorbed into a living system or is made available at the site of physiological activity.

A chimeric protein, as used herein, refers to any protein comprised of a first amino acid sequence derived from a first source, bonded, covalently or non-covalently, to a second amino acid sequence derived from a second source, wherein the first and second source are not the same. A first source and a second source that are not the same can include two different biological entities, or two different proteins from the same biological entity, or a biological entity and a non-biological entity. A chimeric protein can include for example, a protein derived from at least two different biological sources. A biological source can include any non-synthetically produced nucleic acid or amino acid sequence (e.g., a genomic or cDNA sequence, a plasmid or viral vector, a native virion or a mutant or analog, as further described herein, of any of the above). A synthetic source can include a protein or nucleic acid sequence produced chemically and not by a biological system (e.g., solid phase synthesis of amino acid sequences). A chimeric protein can also include a protein derived from at least two different synthetic sources or a protein derived from at least one biological source and at least one synthetic source. A chimeric protein may also comprise a first amino acid sequence derived from a first source, covalently or non-covalently linked to a nucleic acid, derived from any source or a small organic or inorganic molecule derived from any source. The chimeric protein may comprise a linker molecule between the first and second amino acid sequence or between the first amino acid sequence and the nucleic acid, or between the first amino acid sequence and the small organic or inorganic molecule.

DNA Construct, as used herein, means a DNA molecule, or a clone of such a molecule, either single- or double-stranded that has been modified through human intervention to contain segments of DNA combined in a manner that as a whole would not otherwise exist in nature. DNA constructs contain the information necessary to direct the expression of polypeptides of interest. DNA constructs can include promoters, enhancers and transcription terminators. DNA constructs containing the information necessary to direct the secretion of a polypeptide will also contain at least one secretory signal sequence.

A fragment, as used herein, refers to a peptide or polypeptide comprising an amino acid sequence of at least 2 contiguous amino acid residues, of at least 5 contiguous amino acid residues, of at least 10 contiguous amino acid residues, of at least 15 contiguous amino acid residues, of at least 20 contiguous amino acid residues, of at least 25 contiguous amino acid residues, of at least 40 contiguous amino acid residues, of at least 50 contiguous amino acid residues, of at least 100 contiguous amino acid residues, or of at least 200 contiguous amino acid residues or any deletion or truncation of a protein, peptide, or polypeptide.

Linked, as used herein, refers to a first nucleic acid sequence covalently joined to a second nucleic acid sequence. The first nucleic acid sequence can be directly joined or juxtaposed to the second nucleic acid sequence or alternatively an intervening sequence can covalently join the first sequence to the second sequence. Linked as used herein can also refer to a first amino acid sequence covalently joined to a second amino acid sequence. The first amino acid sequence can be directly joined or juxtaposed to the second amino acid sequence or alternatively an intervening sequence can covalently join the first amino acid sequence to the second amino acid sequence. Linked as used herein can also refer to a first amino acid sequence covalently joined to a nucleic acid sequence or a small organic or inorganic molecule.

Operatively linked, as used herein, means a first nucleic acid sequence linked to a second nucleic acid sequence such that both sequences are capable of being expressed as a biologically active protein or peptide.

Polypeptide, as used herein, refers to a polymer of amino acids and does not refer to a specific length of the product; thus, peptides, oligopeptides, and proteins are included within the definition of polypeptide. This term does not exclude post-expression modifications of the polypeptide, for example, glycosylation, acetylation, phosphorylation, pegylation, addition of a lipid moiety, or the addition of any organic or inorganic molecule. Included within the definition, are for example, polypeptides containing one or more analogs of an amino acid (including, for example, unnatural amino acids) and polypeptides with substituted linkages, as well as other modifications known in the art, both naturally occurring and non-naturally occurring.

High stringency, as used herein, includes conditions readily determined by the skilled artisan based on, for example, the length of the DNA. Generally, such conditions are set forth by Sambrook et al. Molecular Cloning: A Laboratory Manual, 2 ed. Vol.1, pp.1.101-104, Cold Spring Harbor Laboratory Press, (1989), and include use of a prewashing solution for the nitrocellulose filters 5X SSC, 0.5% SDS, 1.0 mM EDTA (PH 8.0), hybridization conditions of 50% formamide, 6X SSC at 42° C. (or other similar hybridization solution, such as Stark's solution, in 50% formamide at 42° C.), and with washing at approximately 68° C., 0.2 times SSC, 0.1% SDS. The skilled artisan will recognize that the temperature and wash solution salt concentration can be adjusted as necessary according to factors such as the length of the probe.

Moderate stringency, as used herein, includes conditions that can be readily determined by those having ordinary skill in the art based on, for example, the length of the DNA. The basic conditions are set forth by Sambrook et al. Molecular Cloning: A Laboratory Manual, 2 ed. Vol.1, pp. 1.101-104, Cold Spring Harbor Laboratory Press, (1989), and include use of a prewashing solution for the nitrocellulose filters 5×SSC, 0.5% SDS, 1.0 mM EDTA (PH 8.0), hybridization conditions of 50% formamide, 6×SSC at 42° C. (or other similar hybridization solution, such as Stark's solution, in 50% formamide at 42° C.), and washing conditions of 60° C., 0.5X SSC, 0.1% SDS.

A small inorganic molecule, as used herein means a molecule containing no carbon atoms and being no larger than 50 kD.

A small organic molecule, as used herein means a molecule containing at least one carbon atom and being no larger than 50 kD.

Treat, treatment, treating, as used herein means, any of the following: the reduction in severity of a disease or condition; the reduction in the duration of a disease course; the amelioration of one or more symptoms associated with a disease or condition; the provision of beneficial effects to a subject with a disease or condition, without necessarily curing the disease or condition, the prophylaxis of one or more symptoms associated with a disease or condition.

Unbound, as used herein, refers to a first molecule that does not become associated with a second molecule, either covalently or non-covalently, subsequent to administration of the molecule to a subject.

B. Serum Albumin Binding

The chimeric protein of the invention comprises a modified biologically active molecule that binds less serum albumin compared to a biologically active molecule not so modified. The serum albumin can be serum albumin of any mammal, e.g., human, non-human primate, porcine, bovine, murine or rat albumin. In a specific embodiment the albumin is human albumin.

1. Measuring Serum Albumin Binding

Many methods known in the art can be used to measure serum albumin binding, e.g., surface plasmon resonance (BIACORE™ Biacore AB, Piscataway, N.J.) size exclusion chromatography, equilibrium dialysis, ultra-filtration or analytical ultra-centrifugation (see, e.g., Oravcova et al. 1996, J. Chromatogr. 677:1; Hage et al. 1997, J Chromatogr. 699:499; Frostell-Karlson et al. 2000, J. Med. Chem. 43:1986).

Serum albumin binding can be measured using biosensor technology, e.g., surface plasmon resonance (Frostell-Karisson et al. 2000, J. Med. Chem. 43: 1986). In this method, serum albumin can be immobilized on a solid support, e.g., a chip. The sensor chip is placed in contact with an integrated fluidic cartridge (IFC) and a detection unit. Continuous buffer flows through the IFC and over the chip surface. A sample molecule of interest is injected over the surface, using an autoinjector and refractive index changes, as a result of binding events close to the surface, are detected by the detection unit. Such automated devices are well known in the art (e.g., Biacore 3000, Biacore AB, Uppsala, Sweden). Compounds can be injected at a single concentration and compared to that of a selected reference compound. The advantages of biosensor technology are that binding is monitored directly without the use of labels, sample consumption is low, and analysis is rapid and automated.

More conventional means, such as equilibrium dialysis or ultrafiltration can be used to separate, detect and/or measure serum albumin binding to a molecule of interest. Equilibrium dialysis is based on establishment of an equilibrium state between a protein compartment and a buffer compartment, which are separated by a membrane that is permeable only for a low-molecular weight species. Ultrafiltration uses semipermeable membranes under a pressure gradient to achieve separation of complexes of serum albumin and a molecule of interest and unbound species. Ultracentrifugation can also be used to separate, detect and/or measure serum albumin binding to a molecule of interest. Ultracentrifugation does not rely on a membrane, but instead relies solely on centrifugal force to achieve separation of bound and unbound species.

Various chromatographic methods can be used to separate, detect and/or measure serum albumin binding to a molecule of interest. Affinity chromatography can be used, where serum albumin is immobilized on a solid support. If this method is used care must be taken to insure that the immobilization does not influence serum albumin binding properties. This can be determined by running known standards with established affinity for serum albumin and comparing the binding to immobilized serum albumin with serum albumin in solution.

Size exclusion chromatography can be used to separate, detect and/or measure serum albumin binding to a molecule of interest. A sample containing a molecule of interest and serum albumin can be directly applied to a size exclusion column. Larger species elute quickly, i.e. complexes of serum albumin and the molecule of interest, while unbound species are retained on the column longer. Dissociation constants and association constants must be considered when using this technique. Rapidly associating/dissociating species may affect accuracy where the goal is to determine how much of a molecule of interest binds serum albumin.

Size exclusion chromatography can be combined with reverse phase chromatography. In this system larger complexes flow though the column in the void volume. Smaller molecules enter into pores in the column matrix material. The matrix material can be functionalized (e.g., with a tripeptide Gly-Phe-Phe) which will interact with the molecule of interest through hydrophobic interactions causing it to be retained, thus providing greater separation of the species.

Electrophoretic techniques can also be used to separate, detect and/or measure serum albumin binding to a molecule of interest. The serum albumin can be soluble or immobilized on the matrix material. Binding can be detected as gel shift of a band indicating higher molecular weight. This, of course, requires the use of a label such as a radioactive label. Capillary electrophoresis can be used. In this method samples are directly applied to small capillary tubes containing an electrophoretic matrix. This method can be combined with affinity separation whereby the serum albumin is immobilized within the matrix. Alternatively, the serum albumin can be placed in the electrophoresis running buffer.

C. Chimeric Proteins Comprising Modified Biologically Active Molecules

Obtaining and sustaining pharmacologically effective levels of biologically active molecules, e.g., therapeutics, is a challenge in the treatment of most diseases and conditions requiring drug therapy. One of the most daunting problems associated with maintaining sustained effective serum concentrations of biologically active molecules is the binding of the biologically active molecule to circulating serum proteins such as albumin. Drug-serum albumin binding effectively limits the amount of a biologically active molecule that is capable of reaching its target and acting in an efficacious manner (e.g., binding a target cell or molecule). The invention is based on the surprising discovery that by modifying a biologically active molecule by linking it to an Fc fragment of an immunoglobulin binding of the biologically active molecule to serum albumin can be prevented or inhibited, thus providing for a controllable sustained unbound serum level of the biologically active molecule. In one embodiment, the invention thus relates to a chimeric protein comprising a biologically active molecule having a modification, wherein said modification comprises linking said biologically active molecule to at least a portion of an immunoglobulin constant region, and wherein said biologically active molecule binds less serum albumin compared to the same biologically active molecule without said modification. In another embodiment the chimeric protein comprising the modified biologically active molecule binds substantially no serum albumin. Substantially no serum albumin binding means serum albumin binding has been reduced by at least 80%, at least 90%, at least 95%, at least 99% compared to the biologically active molecule not modified to comprise at least a portion of an immunoglobulin constant region. The portion of the immunoglobulin may be an Fc fragment, or a portion that binds FcRn.

In discussion of this invention, reference will be made to “serum albumin,” but the invention envisions that such chimeric proteins may optionally have less binding, or no binding, to human serum albumin.

1. Structure of Chimeric Proteins Comprising Modified Biologically Active Molecules

The chimeric protein of the invention comprises at least one biologically active molecule, at least a portion of an immunoglobulin constant region, and optionally a linker. In certain embodiments, the portion of the immunoglobulin may be an Fc fragment, or a portion that binds FcRn. While embodiments of the invention will be presented with an Fc fragment, one skilled in the art could substitute at least a portion of an immunoglobulin constant region, or at least the FcRn binding portion of an immunoglobulin constant region in any of the examples or particular embodiments defined in this application.

The Fc fragment of an immunoglobulin will have both an N, or an amino terminus, and a C, or carboxy terminus. The chimeric protein of the invention may have the biologically active molecule linked to the N terminus of the Fc fragment of an immunoglobulin. The biologically active molecule may be linked to the C terminus of the portion of an immunoglobulin constant region. Alternatively, the biologically active molecule is not linked to either terminus, but is instead linked to a position contained between the two termini. In one embodiment, the linkage is a covalent bond. In another embodiment, the linkage is a non-covalent association.

The chimeric protein can optionally comprise at least one linker, thus the biologically active molecule does not have to be directly linked to the Fc fragment of an immunoglobulin. The linker can intervene in between the biologically active molecule and the Fc fragment of an immunoglobulin. The linker can be linked to the N terminus of the Fc fragment of an immunoglobulin, or the C terminus of the Fc fragment of an immunoglobulin. When the biologically active molecule is a polypeptide, or fragment of any of the preceding, it will have both an N terminus and a C terminus. The linker can be linked to the N terminus of the biologically active molecule, or the C terminus of the biologically active molecule.

The invention thus relates to a chimeric protein comprising at least one biologically active molecule (X), optionally, a linker (L), and at least one Fc fragment of an immunoglobulin (F). In one embodiment, the invention relates to a modified biologically active molecule comprised of the formula
X-L-F
wherein X is linked at its C terminus to the N terminus of L, and L is a direct link or a linker linked at its C terminus to the N terminus of F

In another embodiment, the invention relates to a modified biologically active molecule comprised of the formula
F-L-X
wherein F is linked at its C terminus to the N terminus of L, and L is a direct link or a linker linked at its C terminus to the N terminus of X.

The chimeric protein of the invention includes monomers, dimers, as well higher order multimers. In one embodiment, the chimeric protein is a monomer comprising one biologically active molecule and one Fc fragment of an immunoglobulin. In another embodiment, the chimeric protein of the invention is a dimer comprising two biologically active molecules and two Fc fragments of an immunoglobulin. In one embodiment, the two biologically active molecules are the same. In one embodiment, the two biologically active molecules are different. In one embodiment, the two Fc fragments of an immunoglobulin are the same. In another embodiment, the modified biologically active molecule is a heterodimer comprising a first chain and a second chain, wherein said first chain comprises an Fc fragment of an immunoglobulin linked to a biologically active molecule and said second chain comprises an Fc fragment of an immunoglobulin without a biologically active molecule linked to it.

Such modified biologically active molecules may be described using the formulas set forth in Table 1, where 1, L, and F are as described above, and where (') indicates a different molecule than without (') and where (:)indicates a non-peptide bond.

TABLE 1

X-F:F-X
X′-F:F-X
X-L-F:F-X
X-F:F-L-X
X-L-F:F-L-X
X′-L-F:F-L-X
X-L′-F:F-L-X
X′-L′-F:F-L-X
F:F-X
F:F-L-X
X-F:F
X-L-F:F
L-F:F-X
X-F:F-L

The skilled artisan will understand additional combinations are possible including the use of additional linkers and these are encompassed by the present invention.

2. Biologically Active Molecules

The invention contemplates the use of any biologically active molecule in the chimeric protein of the invention. The biologically active molecule can include a protein, a peptide, and/or a polypeptide, including fragments of any of the preceding. The biologically active molecule can be a single amino acid. The biologically active molecule can include a modified protein, peptide or polypeptide, including fragments of any of the preceding. The modification can include, but is not limited to glycosylation, the addition of a lipid moiety, pegylation, or a modification with any other organic or inorganic molecule. The polypeptide, or fragment thereof, can be comprised of at least one non-naturally occurring amino acid.

The biologically active molecule can include a lipid molecule (e.g., a steroid or cholesterol, a fatty acid, a triacylglycerol, glycerophospholipid, or sphingolipid). The biologically active molecule can include a sugar molecule (e.g., glucose, sucrose, mannose). The biologically active molecule can include a nucleic acid molecule (e.g., DNA, RNA). The biologically active molecule can include a small organic or inorganic molecule (see, e.g., U.S. Pat. Nos. 6,086,875, 6,030,613, 6,485,726, PCT Application No. US/02/21335).

a. Antiviral Agents

In one embodiment, the biologically active molecule is an antiviral agent. An antiviral agent can include any molecule that inhibits or prevents viral replication, or inhibits or prevents viral entry into a cell, or inhibits or prevents viral egress from a cell. In one embodiment, the antiviral agent is a fusion inhibitor.

The viral fusion inhibitor for use in the chimeric protein of the invention can be any molecule that decreases or prevents viral penetration of a cellular membrane of a target cell. The viral fusion inhibitor can be any molecule that decreases or prevents the formation of syncytia between at least two susceptible cells. The viral fusion inhibitor can be any molecule that decreases or prevents the joining of a lipid bilayer membrane of a eukaryotic cell and a lipid bilayer of an enveloped virus. Examples of enveloped virus include, but are not limited to HIV-1, HIV-2, SIV, influenza, parainfluenza, Epstein-Barr virus, CMV, herpes simplex 1, herpes simplex 2, SARS virus and respiratory syncytia virus (see, e.g., U.S. Pat. Nos. 6,086,875, 6,030,613, 6,485,726 PCT Application No. US/02/21335).

The viral fusion inhibitor can be any molecule that decreases or prevents viral fusion. In one embodiment, the viral fusion inhibitor is a peptide of 3-36 amino acids, 3-45 amino acids, 10-50 amino acids, or 20-65 amino acids. The peptide can be comprised of a naturally occurring amino acid sequence (e.g., a fragment of gp41) including analogs and mutants thereof or the peptide can be comprised of an amino acid sequence not found in nature, so long as the peptide exhibits viral fusion inhibitory activity.

In one embodiment, the viral fusion inhibitor is a protein, a protein fragment, a peptide, a peptide fragment identified as being a viral fusion inhibitor using at least one computer algorithm, e.g., ALLMOTI5, 107×178×4 and PLZIP (see, e.g., U.S. Pat. Nos. 6,013,263, 6,015,881, 6,017,536, 6,020,459, 6,060,065, 6,068,973, 6,093,799 and 6,228,983).

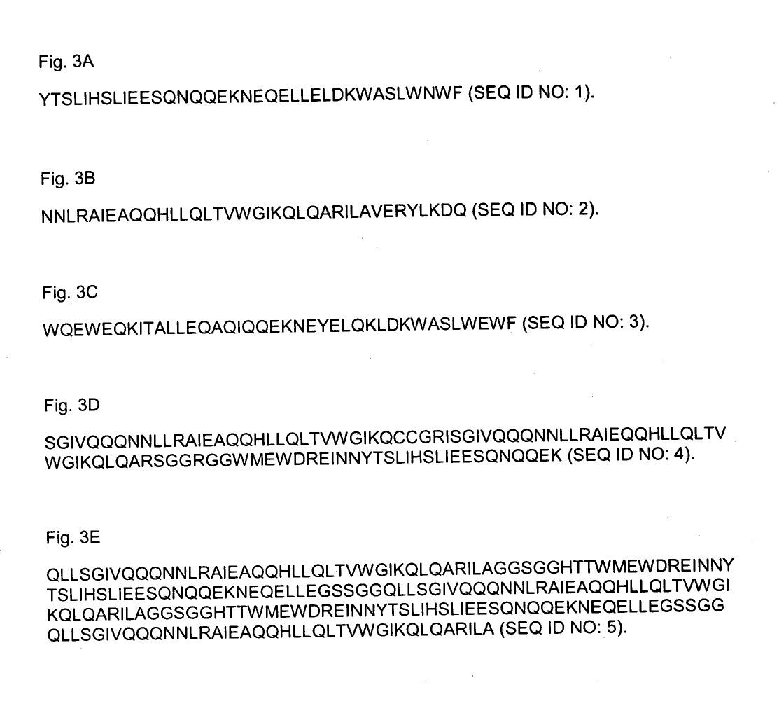

In one embodiment, the viral fusion inhibitor is an HIV fusion inhibitor. In one embodiment, HIV is HIV-1. In another embodiment, HIV is HIV-2. In one embodiment, the HIV fusion inhibitor is a peptide comprised of a fragment of the gp41 envelope protein of HIV-1. The HIV fusion inhibitor can comprise, e.g., T20 (SEQ ID NO: 1) (FIG. 3A) or an analog thereof, T21 (SEQ ID NO: 2) (FIG. 3B) or an analog thereof, T1249 (SEQ ID NO: 3) (FIG. 3C) or an analog thereof, N_{CCGg}P41 (SEQ ID NO: 4) (FIG. 3D) (Louis et al. 2001 J. Biol. Chem. 276(31):29485)) or an analog thereof, or 5 helix (SEQ ID NO: 5) (FIG. 3E) (Root et al. 2001, Science 291:884) or an analog thereof.

Assays known in the art can be used to test for antiviral activity of a molecule, e.g., viral fusion inhibiting activity of a protein, a protein fragment, a peptide, a peptide fragment, a small organic molecule, or a small inorganic molecule. These assays include a reverse transcriptase assay, a p24 assay, or syncytia formation assay (see, e.g., U.S. Pat. No. 9,464,933).

b. Other Proteinaceous Biologically Active Molecules

In one embodiment, the biologically active molecule comprises a growth factor, hormone, cytokine, or analog or fragment thereof. In another embodiment, the biologically active molecule comprises a molecule having the activity of a growth factor hormone, or cytokine or an analog of a growth factor hormone. In one embodiment, biologically active molecule is an analog of leutinizing releasing hormone (LHRH), e.g., leuprolide. The biologically active molecule can include, but is not limited to, erythropoietin (EPO), RANTES, MIP1α, MIP1β, IL-2, IL-3, GM-CSF, growth hormone, tumor necrosis factor (e.g., TNFα or β), interferon α, interferon β, epidermal growth factor, follicle stimulating hormone, progesterone, estrogen, or testosterone (see, e.g., U.S. Pat. Nos. 6,086,875, 6,030,613, 6,485,726 PCT Application No. US/02/21335).

In one embodiment, biologically active molecule comprises a receptor, or a fragment, or analog thereof. The receptor can be expressed on a cell surface, or alternatively the receptor can be expressed on the interior of the cell. The receptor can be a viral receptor, e.g., CD4, CCR5, CXCR4, CD21, and CD46. The receptor can be a bacterial receptor. The biologically active molecule can be an extra-cellular matrix protein or fragment or analog thereof, important in bacterial colonization and infection (see, e.g., U.S. Pat. Nos. 5,648,240, 5,189,015, 5,175,096) or a bacterial surface protein important in adhesion and infection (see, e.g., U.S. Pat. No. 5,648,240). The biologically active molecule can be a growth factor, hormone or cytokine receptor, or a fragment or analog thereof, e.g., TNFα receptor, the erythropoietin receptor, CD25, CD122, CD132. Also included are molecules having receptor like activity, i.e. able to bind a ligand of a receptor.

C. Nucleic Acids

In one embodiment, the biologically active molecule is a nucleic acid, e.g., DNA, RNA. In one specific embodiment the biologically active molecule is a nucleic acid that can be used in RNA interference (RNAi). The nucleic acid molecule can be as an example, but not as a limitation, an anti-sense molecule or a ribozyme.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarily, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo); agents facilitating transport across the cell membrane (see, e.g., Letsinger, et al. 1989, Proc. Natl. Acad. Sci. USA 86:6553; Lemaitre, et al. 1987, Proc. Natl. Acad. Sci. USA 84:648; PCT Publication No. WO 88/09810) or the blood-brain barrier (see, e.g., PCT Publication No. WO 89/10134); hybridization-triggered cleavage agents (see, e.g., Krol et al. 1988, BioTechniques 6:958); or intercalating agents (see, e.g., Zon, 1988, Pharm. Res. 5:539). To this end, the oligonucleotide may be conjugated to another molecule, e.g., a peptide, hybridization triggered cross-linking agent, transport agent, or hybridization-triggered cleavage agent.

Ribozyme molecules designed to catalytically cleave target gene mRNA transcripts can also be used to prevent translation of target gene mRNA and, therefore, expression of target gene product (see, e.g., PCT Publication No. WO 90/11364; Sarver, et al., 1990, Science 247,1222-1225).

Ribozymes are enzymatic RNA molecules capable of catalyzing the specific cleavage of RNA (see Rossi, 1994, Current Biology 4:469). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246.

In one embodiment, ribozymes that cleave mRNA at site-specific recognition sequences can be used to destroy target gene mRNAs. In another embodiment, the use of hammerhead ribozymes is contemplated. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, and in Haseloff and Gerlach, 1988, Nature, 334:585.

d. Small Molecules

In one embodiment the biologically active molecule is a small molecule (see, e.g., U.S. Pat. Nos. 6,086,875; 6,030,613; 6,485, 726; and PCT Application No. US/02/21335). A small molecule can include any organic or inorganic molecule no larger than 50 kD administered as a therapeutic. The small molecule, in certain embodiments, may be no larger than: 45 kD, 40 kD, 35 kD, 30 kD, 25 kD, 20 kD, 15 kD, 10 kD, or 5 kD. Many small molecules are known in the art for treatment of different diseases and any of these could be used in the invention. Examples include, but are not limited to salbutamol, quinine, rifampicin, ketanserin, tolterodine, prednisone, diazepam, salicylic acid, phenyloin, coumarin, sulfadimethoxine, pyrimetamie, digitoxin, warfarin and naproxen.

3. Immunoglobulins

The chimeric proteins of this invention include at least a portion of an immunoglobulin constant region. Immunoglobulins are comprised of four protein chains that associate covalently—two heavy chains and two light chains. Each chain is further comprised of one variable region and one constant region. Depending upon the immunoglobulin isotype, the heavy chain constant region is comprised of 3 or 4 constant region domains (e.g., CH₁, CH₂, CH₃, CH₄). Some isotypes are further comprised of a hinge region.

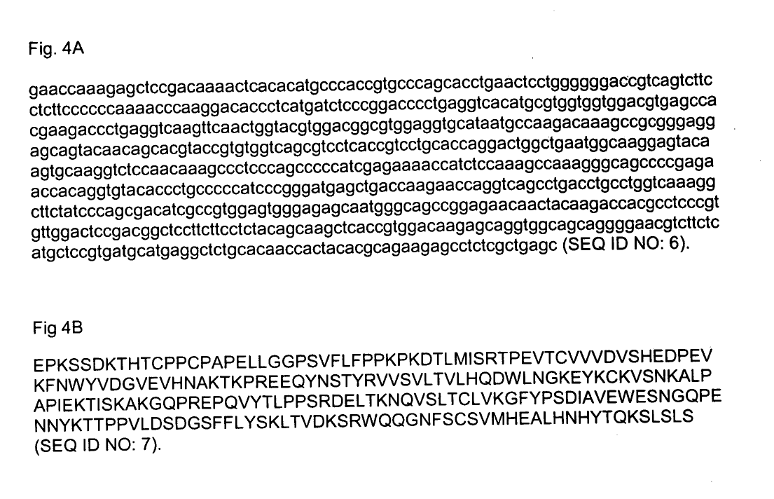

The chimeric protein of the invention can comprise an Fc fragment or analog thereof. An Fc fragment can be comprised of the CH2 and CH3 domains of an immunoglobulin and the hinge region of the immunoglobulin. The Fc fragment can be the Fc fragment of an IgG1, an IgG2, an IgG3 or an IgG4. In one embodiment, the immunoglobulin is an Fc fragment of an IgG1. In another embodiment, the portion of an immunoglobulin constant region is comprised of the amino acid sequence of SEQ ID NO: 6 (FIG. 4A) or an analog thereof. In another embodiment, the immunoglobulin is comprised of a protein, or fragment thereof, encoded by the nucleic acid sequence of SEQ ID NO: 7 (FIG. 4B).

The Fc fragment of an immunoglobulin can be an Fc fragment of an immunoglobulin obtained from any mammal. The Fc fragment of an immunoglobulin can include, but is not limited to, a portion of a human immunoglobulin constant region, a non-human primate immunoglobulin constant region, a bovine immunoglobulin constant region, a porcine immunoglobulin constant region, a murine immunoglobulin constant region, an ovine immunoglobulin constant region or a rat immunoglobulin constant region.

The immunoglobulin can be produced recombinantly or synthetically. The immunoglobulin can be isolated from a cDNA library. The immunoglobulin can be isolated from a phage library (see, e.g., McCafferty et al. 1990, Nature 348: 552). The immunoglobulin can be obtained by gene shuffling of known sequences (Mark et al., 1992, Bio/Technol. 10: 779). The immunoglobulin can be isolated by in vivo recombination (Waterhouse et al., 1993, Nucl. Acid Res. 21:2265). The immunoglobulin can be a humanized immunoglobulin (Jones et al., 1986, Nature 332: 323).

The portion of an immunoglobulin constant region can include at least one of at least a portion of an IgG, an IgA, an IgM, an IgD, and an IgE. In one embodiment, the immunoglobulin is an IgG. In another embodiment, the immunoglobulin is IgG1. In another embodiment, the immunoglobulin is IgG2.

In another embodiment, the portion of an immunoglobulin constant region is an Fc neonatal receptor (FcRn) binding partner. An FcRn binding partner is any molecule that can be specifically bound by the FcRn receptor with consequent active transport by the FcRn receptor of the FcRn binding partner. Specifically bound refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant K_{A} is higher than 10⁶M⁻¹, or more preferably higher than 108 M⁻¹. If necessary, non-specific binding can be reduced without substantially affecting specific binding by varying the binding conditions. The appropriate binding conditions such as concentration of the molecules, ionic strength of the solution, temperature, time allowed for binding, concentration of a blocking agent (e.g., serum albumin, milk casein), etc., may be optimized by a skilled artisan using routine techniques.

The FcRn receptor has been isolated from several mammalian species including humans. The sequences of the human FcRn, rat FcRn, and mouse FcRn are known (Story et al. 1994, J. Exp. Med. 180:2377). The FcRn receptor binds IgG (but not other immunoglobulin classes such as IgA, IgM, IgD, and IgE) at relatively low pH, actively transports the IgG transcellularly in a luminal to serosal direction, and then releases the IgG at relatively higher pH found in the interstitial fluids. It is expressed in adult epithelial tissue (U.S. Pat. Nos. 6,030,613 and 6,086,875) including lung and intestinal epithelium (Israel et al. 1997, Immunology 92:69) renal proximal tubular epithelium (Kobayashi et al. 2002, Am. J. Physiol. Renal Physiol. 282:F358) as well as nasal epithelium, vaginal surfaces, and biliary tree surfaces.

FcRn binding partners of the present invention encompass any molecule that can be specifically bound by the FcRn receptor including whole IgG, the Fc fragment of IgG, and other fragments that include the complete binding region of the FcRn receptor. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. The major contact sites include amino acid residues 248, 250-257, 272, 285, 288, 290-291, 308-311, and 314 of the CH2 domain and amino acid residues 385-387, 428, and 433-436 of the CH3 domain. Fc-FcRn contacts are all within a single Ig heavy chain. Two FcRn receptors can bind a single Fc molecule. Crystallographic data suggest that each FcRn molecule binds a single polypeptide of the Fc homodimer. References made to amino acid numbering of immunoglobulins or immunoglobulin fragments, or regions, are all based on Kabat et al. 1991, Sequences of Proteins of Immunological Interest, U.S. Department of Public Health, Bethesda, Md.

The Fc region of IgG can be modified according to well recognized procedures such as site directed mutagenesis and the like to yield modified IgG or Fc fragments or portions thereof that will be bound by FcRn. Such modifications include modifications remote from the FcRn contact sites as well as modifications within the contact sites that preserve or even enhance binding to the FcRn. For example the following single amino acid residues in human IgG1 Fc (Fcγ1) can be substituted without significant loss of Fc binding affinity for FcRn: P238A, S239A, K246A, K248A, D249A, M252A, T256A, E258A, T260A, D265A, S267A, H268A, E269A, D270A, E272A, L274A, N276A, Y278A, D280A, V282A, E283A, H285A, N286A, T289A, K290A, R292A, E293A, E294A, Q295A, Y296F, N297A, S298A, Y300F, R301A, V303A, V305A, T307A, L309A, Q311A, D312A, N315A, K317A, E318A, K320A, K322A, S324A, K326A, A327Q, P329A, A330Q, P331A, E333A, K334A, T335A, S337A, K338A, K340A, Q342A, R344A, E345A, Q347A, R355A, E356A, M358A, T359A, K360A, N361A, Q362A, Y373A, S375A D376A, A378Q, E380A, E382A, S383A,N384A, Q386A, E388A, N389A, N390A, Y391F, K392A, L398A, S400A, D401A, D413A, K414A, R416A, Q418A, Q419A, N421A, V422A, S424A, E430A, N434A, T437A, Q438A, K439A, S440A, S444A, and K447A, where for example P238A represents wildtype proline substituted by alanine at position 238. In addition to alanine other amino acids may be substituted for the wildtype amino acids at the positions specified above. Mutations may be introduced singly into Fc giving rise to more than one hundred FcRn binding partners distinct from native Fc. Additionally, combinations of two, three, or more of these individual mutations may be introduced together, giving rise to hundreds more FcRn binding partners, see Kabat et al. 1991, Sequences of Proteins of Immunological Interest, U.S. Department of Public Health, Bethesda, Md.

Certain of the above mutations may confer new functionality upon the FcRn binding partner. For example, one embodiment incorporates N297A, removing a highly conserved N-glycosylation site. The effect of this mutation is to reduce immunogenicity, thereby enhancing circulating half life of the FcRn binding partner, and to render the FcRn binding partner incapable of binding to FcyRI, FcyRIIA, FcyRIIB, and FcyRIIIA, without compromising affinity for FcRn (Routledge et al. 1995, Transplantation 60:847; Friend et al. 1999, Transplantation 68:1632; Shields et al. 1995, J. Biol. Chem. 276:6591). Additionally, at least three human Fc gamma receptors appear to recognize a binding site on IgG within the lower hinge region, generally amino acids 234-237. Therefore, another example of new functionality and potential decreased immunogenicity may arise from mutations of this region, as for example by replacing amino acids 233-236 of human IgG1 “ELLG” to the corresponding sequence from IgG2 “PVA” (with one amino acid deletion). It has been shown that FcyRl, FcyR11, and FcyRIII, which mediate various effector functions, will not bind to IgG1 when such mutations have been introduced (Ward and Ghetie 1995, Therapeutic Immunology 2:77 and Armour et al. 1999, Eur. J. Immunol. 29:2613). As a further example of new functionality arising from mutations described above affinity for FcRn may be increased beyond that of wild type in some instances. This increased affinity may reflect an increased “on” rate, a decreased “off” rate or both an increased “on” rate and a decreased “off” rate. Mutations believed to impart an increased affinity for FcRn include T256A, T307A, E380A, and N434A (Shields et al. 2001, J. Biol. Chem. 276:6591).

In one embodiment the FcRn binding partner is a polypeptide including the sequence PKNSSMISNTP (SEQ ID NO: 8) and optionally further including a sequence selected from the HQSLGTQ (SEQ ID NO: 9), HQNLSDGK (SEQ ID NO: 10), HQNISDGK (SEQ ID NO: 11), or VISSHLGQ (SEQ ID NO: 12) (U.S. Pat. No. 5,739,277).

The skilled artisan will understand that portions of an immunoglobulin constant region for use in the chimeric protein of the invention can include mutants or analogs thereof, or can include chemically modified (e.g. pegylation) immunoglobulin constant regions or fragments thereof (see, e.g., Aslam and Dent 1998, Bioconjugation: Protein Coupling Techniques For the Biomedical Sciences Macmilan Reference, London). In one instance a mutant can provide for enhanced binding of an FcRn binding partner for the FcRn. Also contemplated for use in the chimeric protein of the invention are peptide mimetics of at least a portion of an immunoglobulin constant region, e.g., a peptide mimetic of an Fc fragment or a peptide mimetic of an FcRn binding partner. In one embodiment, the peptide mimetic is identified using phage display (see, e.g., McCafferty et al. 1990, Nature 348:552, Kang et al. 1991, Proc. Natl. Acad. Sci. USA 88:4363; EP 0 589 877 B1).

4. Optional Linkers

The modified biologically active molecule of the invention can optionally comprise at least one linker molecule. The linker can be comprised of any organic molecule. In one embodiment, the linker is polyethylene glycol (PEG). In another embodiment the linker is comprised of amino acids. The linker can comprise 1-5 amino acids, 1-10 amino acids, 1-20 amino acids, 10-50 amino acids, 50-100 amino acids, or 100-200 amino acids. The linker can comprise the sequence Gₙ, wherein n is an integer from 1-10. The linker can comprise the sequence (GGS)ₙ (SEQ ID NO: 13), wherein n is an integer from 1-10. Examples of linkers include, but are not limited to GGG (SEQ ID NO: 14), SGGSGGS (SEQ ID NO: 15), GGSGGSGGSGGSGGG (SEQ ID NO: 16), GGSGGSGGSGGSGGSGGS (SEQ ID NO: 17), and FC. In a specific embodiment the linker is a dendrimer. The linker does not eliminate the activity of the modified biologically active molecule. Optionally, the linker enhances the activity of the modified biologically active molecule, e.g., by diminishing the effects of steric hindrance and making the biologically active molecule more accessible to its target binding site, e.g., a viral protein, gp41.

5. Variants and Derivatives of Chimeric Proteins

Derivatives and analogs of the chimeric proteins of the invention, antibodies against the chimeric proteins of the invention and antibodies against binding partners of the chimeric proteins of the invention are all contemplated, and can be made by altering their amino acid sequences by substitutions, additions, and/or deletions/truncations or by introducing chemical modifications that result in functionally equivalent molecules. It will be understood by one of ordinary skill in the art that certain amino acids in a sequence of any protein may be substituted for other amino acids without adversely affecting the activity of the protein.

Various changes may be made in the amino acid sequences of the biologically active molecules of the invention or DNA sequences encoding therefore without appreciable loss of their biological activity, function, or utility. Derivatives, analogs, or mutants resulting from such changes and the use of such derivatives are within the scope of the present invention. In a specific embodiment, the derivative is functionally active, i.e. capable of exhibiting one or more activities associated with the modified biologically active molecules of the invention. As an example, but not as a limitation, the biologically active molecule can have antiviral activity, e.g., anti HIV activity. Activity can be measured by assays known in the art. For example, where the biologically active molecule is an HIV inhibitor activity can be tested by measuring reverse transcriptase activity using known methods (see, e.g., Barre-Sinoussi et al. 1983, Science 220:868; Gallo et al. 1984, Science 224:500). Alternatively, activity can be measured by measuring viral fusogenic activity (see, e.g., Nussbaum et al. 1994, J. Virol. 68(9):5411).

Substitutes for an amino acid within the sequence may be selected from other members of the class to which the amino acid belongs (see Table 2). Furthermore, various amino acids are commonly substituted with neutral amino acids, e.g., alanine, leucine, isoleucine, valine, proline, phenylalanine, tryptophan, and methionine (see, e.g., MacLennan et al. 1998, Acta Physiol. Scand. Suppl. 643:55-67; Sasaki et al. 1998, Adv. Biophys. 35:1-24).

TABLE 2

| Original | Exemplary | Typical |
|---|---|---|
| Residues | Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, | Ile |
| | Ala, Phe | |
| Lys (K) | Arg, 1,4-Diamino-butyric | Arg |
| | Acid, Gln, Asn | |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, | Leu |
| | Norleucine | |

D. Nucleic Acid Constructs

The invention relates to a nucleic acid construct comprising a nucleic acid sequence encoding the chimeric protein of the invention, said nucleic acid sequence comprising a first nucleic acid sequence encoding, for example, at least one biologically active molecule, operatively linked to a second nucleic acid sequence encoding an Fc fragment of an immunoglobulin. The nucleic acid sequence can also include additional sequences or elements known in the art (e.g., promoters, enhancers, poly A sequences, signal sequence). The nucleic acid sequence can optionally include a sequence encoding a linker placed between the nucleic acid sequence encoding at least one biologically active molecule and the portion of the immunoglobulin constant region. The nucleic acid sequence can optionally include a linker sequence placed before or after the nucleic acid sequence encoding at least one biologically active molecule and the portion of the immunoglobulin constant region.

In one embodiment, the nucleic acid construct is comprised of DNA. In another embodiment, the nucleic acid construct is comprised of RNA. The nucleic acid construct can be a vector, e.g., a viral vector or a plasmid. Examples of viral vectors include, but are not limited to adeno virus vector, an adeno associated virus vector or a murine leukemia virus vector. Examples of plasmids include but are not limited to, e.g., pUC, pGEM and pGEX.

Due to the known degeneracy of the genetic code, wherein more than one codon can encode the same amino acid, a DNA sequence can vary and still encode a polypeptide having the same amino acid sequence. Such variant DNA sequences can result from silent mutations (e.g., occurring during PCR amplification), or can be the product of deliberate mutagenesis of a native sequence. The invention thus provides isolated DNA sequences encoding polypeptides of the invention, selected from: (a) DNA comprising a nucleotide sequence of a biologically active molecule and an Fc fragment of an immunoglobulin; (b) DNA capable of hybridization to a DNA of (a) under conditions of moderate stringency and which encodes polypeptides of the invention; (c) DNA capable of hybridization to a DNA of (a) under conditions of high stringency and which encodes polypeptides of the invention, and (d) DNA which is degenerate as a result of the genetic code to a DNA defined in (a), (b), or (c), and which encode polypeptides of the invention. Of course, polypeptides encoded by such DNA sequences are encompassed by the invention.

In another embodiment, the nucleic acid molecules of the invention also comprise nucleotide sequences that are at least 80% identical to a native sequence. Also contemplated are embodiments in which a nucleic acid molecule comprises a sequence that is at least 90% identical, at least 95% identical, at least 98% identical, at least 99% identical, or at least 99.9% identical to a native sequence. A native sequence can include any DNA sequence not altered by human intervention. The percent identity may be determined by visual inspection and mathematical calculation. Alternatively, the percent identity of two nucleic acid sequences can be determined by comparing sequence information using the GAP computer program, version 6.0 described by Devereux et al. (Nucl. Acids Res. 12:387,1984) and available from the University of Wisconsin Genetics Computer Group (UWGCG). The preferred default parameters for the GAP program include: (1) a unary comparison matrix (containing a value of 1 for identities and 0 for non identities) for nucleotides, and the weighted comparison matrix of Gribskov and Burgess 1986, Nucl. Acids Res. 14:6745, as described by Schwartz and Dayhoff, eds., Atlas of Protein Sequence and Structure, National Biomedical Research Foundation, pp. 353-358,1979; (2) a penalty of 3.0 for each gap and an additional 0.10 penalty for each symbol in each gap; and (3) no penalty for end gaps. Other programs used by one skilled in the art of sequence comparison may also be used.

E. Synthesis of Modified Biologically Active Molecules

Chimeric proteins comprising an Fc fragment of an immunoglobulin and a biologically active molecule can be synthesized using techniques well known in the art. For example, the modified biologically active molecules of the invention can be synthesized recombinantly in cells (see, e.g., Sambrook et al. 1989, Molecular Cloning A Laboratory. Manual, Cold Spring Harbor Laboratory, N.Y. and Ausubel et al. 1989, Current Protocols in Molecular Biology, Greene Publishing Associates and Wiley Interscience, N.Y.). Alternatively, the modified biologically active molecules of the invention can be synthesized using known synthetic methods such as solid phase synthesis. Synthetic techniques are well known in the art (see, e.g., Merrifield, 1973, Chemical Polypeptides, (Katsoyannis and Panayotis eds.) pp. 335-61; Merrifield 1963, J. Am. Chem. Soc. 85:2149; Davis et al. 1985, Biochem. Intl. 10:394; Finn et al. 1976, The Proteins (3^{rd} ed.) 2:105; Erikson et al. 1976, The Proteins (3^{rd} ed.) 2:257; U.S. Pat. No. 3,941,763). Alternatively, the modified biologically active molecules of the invention can be synthesized using a combination of recombinant and synthetic methods. In certain applications, it may be beneficial to use either a recombinant method or a combination of recombinant and synthetic methods.

Nucleic acids encoding biologically active molecules can be readily synthesized using recombinant techniques well known in the art. Alternatively, the biologically active molecules themselves can be chemically synthesized (see, e.g., U.S. Pat. Nos. 6,015,881; 6,281,331; 6,469,136).

DNA sequences encoding immunoglobulins or fragments thereof may be cloned from a variety of genomic or cDNA libraries known in the art. The techniques for isolating such DNA sequences using probe-based methods are conventional techniques and are well known to those skilled in the art. Probes for isolating such DNA sequences may be based on published DNA sequences (see, for example, Hieter et al., 1980 Cell 22: 197-207). The polymerase chain reaction (PCR) method disclosed by Mullis et al. (U.S. Pat. No. 4,683,195) and Mullis (U.S. Pat. No. 4,683,202) may be used. The choice of library and selection of probes for the isolation of such DNA sequences is within the level of ordinary skill in the art. Alternatively, DNA sequences encoding immunoglobulins or fragments thereof can be obtained from vectors known in the art to contain immunoglobulins or fragments thereof.

For recombinant production, a polynucleotide sequence encoding the modified biologically active molecule is inserted into an appropriate expression vehicle, i.e. a vector that contains the necessary elements for the transcription and translation of the inserted coding sequence, or in the case of an RNA viral vector, the necessary elements for replication and translation. The nucleic acid encoding the modified biologically active molecule is inserted into the vector in proper reading frame.

The expression vehicle is then transfected into a suitable target cell which will express the peptide. Transfection techniques known in the art include, but are not limited to, calcium phosphate precipitation (Wigler et al. 1978, Cell 14:725) and electroporation (Neumann et al. 1982, EMBO, J. 1:841). A variety of host-expression vector systems may be utilized to express the modified biologically active molecule described herein including prokaryotic and eukaryotic cells. These include, but are not limited to, microorganisms such as bacteria (e.g., E. coli) transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems, including mammalian cells (e.g., CHO, Cos, HeLa cells).

The expression vectors can encode for tags that permit for easy purification of the recombinantly produced protein. Examples include, but are not limited to vector pUR278 (Ruther et al. 1983, EMBO J. 2:1791) in which the chimeric protein described herein coding sequence may be ligated into the vector in frame with the lac z coding region so that a hybrid protein is produced. pGEX vectors may also be used to express proteins with a glutathione S-transferase (GST) tag. These proteins are usually soluble and can easily be purified from cells by adsorption to glutathione-agarose beads followed by elution in the presence of free glutathione. The vectors include cleavage sites (thrombin or factor Xa protease or PreScission Protease™ (Pharmacia, Peapack, N.J.) for easy removal of the tag after purification.

Vectors used in transformation will usually contain a selectable marker used to identify transformants. In bacterial systems this can include an antibiotic resistance gene such as ampicillin or kanamycin. Selectable markers for use in cultured mammalian cells include genes that confer resistance to drugs, such as neomycin, hygromycin, and methotrexate. The selectable marker may be an amplifiable selectable marker. One amplifiable selectable marker is the DHFR gene. Another amplifiable marker is the DHFRr cDNA (Simonsen and Levinson 1983, Proc. Natl. Acad. Sci. (USA) 80:2495). Selectable markers are reviewed by Thilly (Mammalian Cell Technology, Butterworth Publishers, Stoneham, Mass.) and the choice of selectable markers is well within the level of ordinary skill in the art.

The chimeric protein of the invention can also be produced by a combination of synthetic chemistry and recombinant techniques. For example, the portion of an immunoglobulin constant region can be expressed recombinantly as described above. The biologically active molecule can be produced using known chemical synthesis techniques (e.g., solid phase synthesis).

The portion of an immunoglobulin constant region can be ligated to the biologically active molecule using appropriate ligation chemistry. For example, the biologically active molecule can be chemically synthesized with an N terminal cysteine. The sequence encoding a portion of an immunoglobulin constant region can be sub-cloned into a vector encoding intein linked to a chitin binding domain. The intein can be linked to the C terminus of the portion of an immunoglobulin constant region. Alternatively, an immunoglobulin constant region can be produced recombinantly with an N terminal cysteine, or the recombinantly produced constant region can be cleaved to reveal an N terminal cysteine. The cysteine can be a native residue (e.g., from an interchain disulfide bridge) or it can be the result of mutational engineering. The biologically active molecule and portion of an immunoglobulin constant region can be reacted together such that nucleophilic rearrangement occurs and the biologically active molecule is covalently linked to the portion of an immunoglobulin constant region via a thio-ester linkage. (Dawsen et al. 2000, Annu. Rev. Biochem. 69:923). The chimeric protein synthesized this way can optionally include a linker peptide between the portion of an immunoglobulin constant region and the viral fusion inhibitor. The linker can for example be synthesized on the N terminus of the biologically active molecule. Linkers can include peptides and/or organic molecules (e.g. polyethylene glycol and/or short amino acid sequences). This combined recombinant and chemical synthesis allows for the rapid screening of chimeric proteins of the invention and linkers to optimize desired properties of the chimeric protein of the invention, e.g., viral fusion inhibitor activity, biological half-life, stability, binding to serum proteins or some other property of the chimeric protein. The method also allows for the incorporation of non-natural amino acids into the chimeric protein of the invention that may be useful for optimizing a desired property of the chimeric protein of the invention. If desired, the chimeric protein produced by this method can be refolded to a biologically active conformation using conditions known in the art, e.g., reducing conditions and then dialyzed slowly into PBS.

F. Methods of Using Chimeric Proteins

The chimeric proteins of the invention have many uses as will be recognized by one skilled in the art, including, but not limited to improved methods of treating a subject with a disease or condition. The improved methods can include providing a chimeric protein comprising a biologically active molecule, e.g., a therapeutic, modified to bind less serum albumin compared to the same biologically active molecule not so modified. The improved methods can include providing a chimeric protein comprising a biologically active molecule, e.g., a therapeutic, modified to bind substantially no serum albumin. Decreasing or eliminating serum albumin binding increases the unbound therapeutically available serum concentration of the biologically active molecule and thus provides for a method of treating a subject that requires lower and less frequent doses, and/or results in fewer associated adverse side effects.

1. Methods of Treating a Patient

The chimeric protein of the invention can be used to prophylactically treat the onset of a disease or condition. Thus, the chimeric protein can be used to treat a subject believed to have been exposed to an infectious agent, e.g., a virus, but who has not yet been positively diagnosed. The chimeric protein can be used to treat a chronic condition such as a chronic viral infection, or an autoimmune disease or an inflammatory condition. Alternatively, the chimeric protein can be used to treat a newly acquired or acute condition such as a non-chronic viral infection or a bacterial infection.
a. Treatment Modalities

The chimeric protein of the invention can be administered intravenously, subcutaneously, intramuscularly, or via any mucosal surface, e.g., orally, sublingually, buccally, nasally, rectally, vaginally or via pulmonary route. The chimeric protein can be implanted within or linked to a biopolymer solid support that allows for the slow release of the chimeric protein to the desired site.

The dose of the chimeric protein of the invention will vary depending on the subject and upon the particular route of administration used. Dosages can range from 0.1 to 100,000 μg/kg body weight. In one embodiment, the dosing range is 0.1-1,000 μg/kg. The chimeric protein can be administered continuously or at specific timed intervals. In vitro assays may be employed to determine optimal dose ranges and/or schedules for administration. For example, where the biologically active molecule is an HIV inhibitor a reverse transcriptase assay, or an rt PCR assay or branched DNA assay can be used to measure HIV concentrations. Additionally, effective doses may be extrapolated from dose-response curves obtained from animal models.

The invention also relates to a pharmaceutical composition comprising a chimeric protein, e.g., at least a portion of an immunoglobulin constant region, a biologically active molecule, and a pharmaceutically acceptable carrier or excipient. Examples of suitable pharmaceutical carriers are described in Remington's Pharmaceutical Sciences by E.W. Martin. Examples of excipients can include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal and sublingual administration the composition may take the form of tablets or lozenges according to conventional protocols.

For administration by inhalation, the compounds for use according to the present invention are conveniently delivered in the form of an aerosol spray from a pressurized pack or nebulizer, with a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can be formulated for parenteral administration (i.e. intravenous or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

The pharmaceutical composition can also be formulated for rectal administration as a suppository or retention enema, e.g., containing conventional suppository bases such as cocoa butter or other glycerides.

2. Methods Of Treating A Patient With Antivirals

In one embodiment, the chimeric protein comprises an antiviral agent. The chimeric protein of the invention prevents or inhibits viral entry into target cells, thereby stopping, preventing, or limiting the spread of a viral infection in a subject and decreasing the viral burden in an infected subject. The invention provides for a chimeric protein which decreases or prevents viral penetration of a cellular membrane of a target cell. The chimeric protein of the invention can prevent the formation of syncytia between at least two susceptible cells. The chimeric protein of the invention can prevent the joining of a lipid bilayer membrane of a eukaryotic cell and an a lipid bilayer of an enveloped virus.

By linking a portion of an immunoglobulin constant region to a viral fusion inhibitor the invention provides a modified biologically active molecule with viral fusion inhibitory activity with little on no serum albumin binding, greater stability and greater bioavailability compared to viral fusion inhibitors alone, e.g., T20, T21, T1249. Thus, in one embodiment, the viral fusion inhibitor decreases or prevents HIV infection of a target cell, e.g., HIV-1.

a. Viral Conditions That May Be Treated

The chimeric protein of the invention can be used to inhibit or prevent the infection of any target cell by any virus. In one embodiment, the virus is an enveloped virus such as, but not limited to HIV, SIV, measles, influenza, Epstein-Barr virus, respiratory syncytia virus, CMV, herpes simplex 1, herpes simplex 2 or parainfluenza virus. In another embodiment, the virus is a non-enveloped virus such as rhino virus or polio virus.

G. Kits

The invention also relates to a kit for measuring serum albumin binding to a molecule of interest. The kit can include a known standard, e.g., a biologically active molecules known to bind serum albumin. The biologically active molecules can be a modified chimeric protein comprising an Fc fragment of an immunoglobulin in a container and an unmodified biologically active molecule in a container. Serum albumin can be provided in a separate container. The molecule of interest can be compared to the standard for serum albumin binding.

### EXAMPLES

### Example 1

### Serum Albumin Binding To Proteins and Therapeutic Peptides

Two molecules of interest were chosen to study the effect the Fc fragment has on serum albumin binding. These included the HIV fusion inhibitor T20, a small peptide, which is administered parentally, and a VLA4 antagonist (Bio 121), which blocks VLA4 adhesion of activated T cells to VCAM on activated endothelium. The VLA4 antagonist was chosen because it is known to bind serum albumin. Chimeric proteins comprised of a molecule of interest and an Fc fragment of an IgG were compared to the same molecule of interest without the Fc fragment for their ability to bind serum albumin.

Analysis of macromolecular interactions was performed using surface plasmon resonance as previously described (Frostell-Karlsson et al. 2000, J. Med. Chem. 43:1986). A BIACORE 3000 instrument (Biacore AB, Piscataway, N.J.) was used and all binding interactions were performed at 25° C. A carboxymethyl-modified dextran (CM5) sensor chip (Biacore AB, Piscataway, N.J.) was used for the analysis. Serum albumin (Albuminar, Aventis, Bridgewater, N.J.) was diluted to 100 μg/mL in 10 mM sodium acetate (pH 4.5) and immobilized to one flowcell of the sensor chip, using amine coupling as described (Frostell-Karlsson et al. 2000, J. Med. Chem. 43:1986). Final immobilization level was approximately 8500 Resonance Units (RU). A “mock-immobilized” surface using a separate flowcell was created using the same procedure in the absence of serum albumin and served as a reference for the binding studies.

Proteins or peptides (analyte) were diluted in HBS-N buffer (10 mM HEPES, pH 7.4; 150 mM NaCl) and injected over the serum albumin and reference surfaces for 3 minutes at a rate of 20 μL/min. After a 35 second dissociation phase, the surface was regenerated by a 30 second pulse of 10 mM glycine (pH 2.0) at a flow rate of 60 μL/min.

The sensorgrams (RU versus time) generated for the mock-coated flowcell were automatically subtracted from the serum albumin-coated sensorgrams. Response at equilibrium (Req) was measured 30 seconds before the end of the injection phase and divided by the molecular weight of the analyte, total response as is in part, a function of molecular weight. (Frostell-Karlsson et al. 2000, J. Med. Chem. 43:1986). Samples tested included T20 linked to Fc (i.e. T20-Fc produced in CHO cells and Fc-T20 produced in E. coli), a VLA4 antagonist linked to Fc, a GnRH peptide linked to Fc, PspA, a bacterial peptide fragment of S. pneumonia surface protein A, peptide YY a peptide involved in regulation of nutrient uptake and an Fc fragment of an immunoglobulin beginning with Cys 226 served as negative controls.

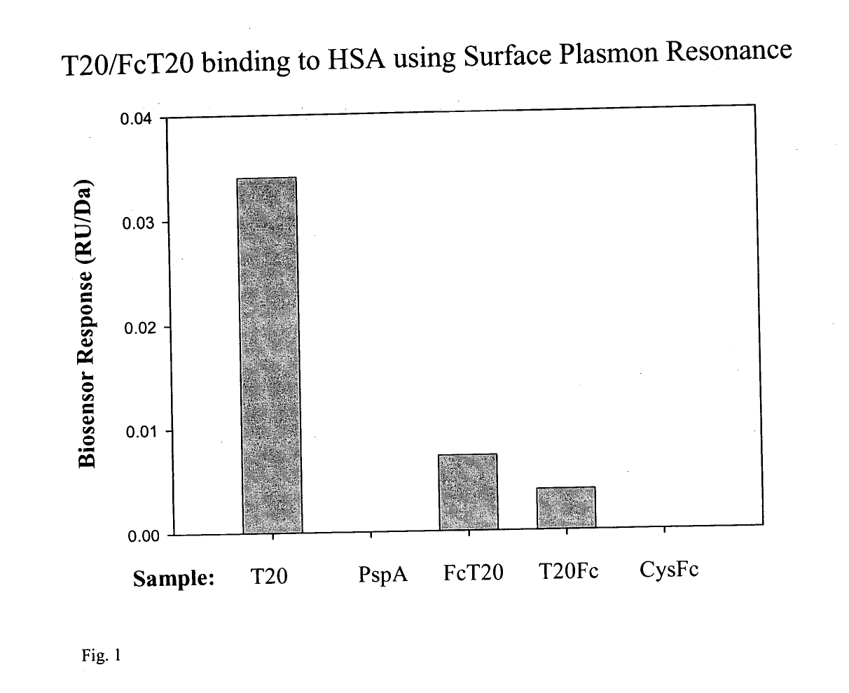

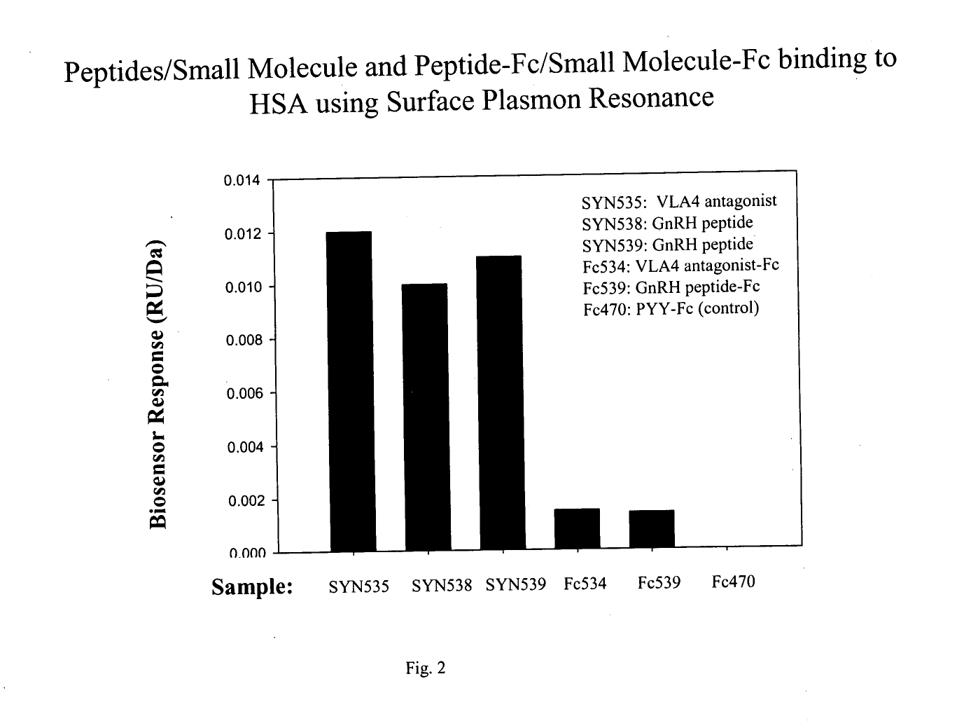

The results demonstrated that human SA bound more than three times as much T20 compared to T20-Fc and Fc-T20, and bound more than 8 times as much VLA4 antagonist compared to VLA4 antagonist-Fc (FIG. 1) and GnRH peptide bound more than 5 times as much HSA compared to GnRH-Fc (FIG. 2). The results are the first demonstration that the Fc fragment of an immunoglobulin can be used to alter the affinity of a molecule of interest for serum albumin, thus providing a method of controlling serum concentrations of therapeutic molecules, which in turn will provide more consistent therapeutic endpoints with fewer unwanted side effects.

### Example 2

### A combination therapy to treat HIV Infection

A patient infected with HIV is treated with a combination of a chimeric protein comprising at least a portion of an immunoglobulin constant region and T20, a viral fusion inhibitor administered sub-cutaneously at 1 mg/kg twice a day in combination with nelfinavir, a protease inhibitor administer at 1 mg/kg twice daily. It is expected that such treatment will result in a lower viral load in the patient compared to administering T20 and nelfinavir alone.

### Example 3

### A Therapy to Treat Prostate Cancer

A patient with prostate cancer is treated with a chimeric protein comprising at least a portion of an immunoglobulin constant region and, leuprolide, an analog of leutenizing hormone releasing hormone (LH-RH) which lowers testosterone levels in patients with advanced prostate cancer and provides palliative relief for the patient. It is administered subcutaneously at 12 μg/day. It is expected that such treatment will result in greater palliative relief in the patient compared to administering leuprolide without a portion of an immunoglobulin constant region.

### Example 4

### Synthesis of CAP-Lys-Asp(OtBu)-Val-Pro-OtBu

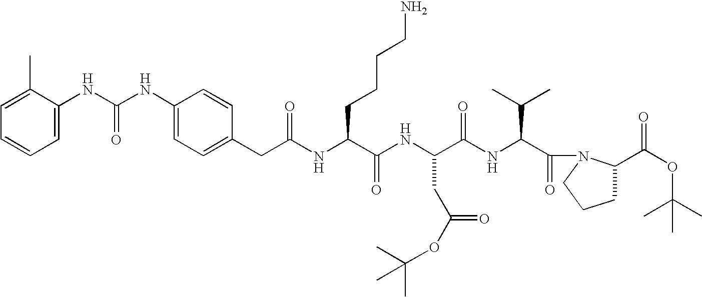

A solution of Cbz-Val-OH (680 mg, 2.70 mmol), H-Pro-OtBu (520 mg (2.50 mmol), DIPEA (870 μl, 5.00 mmol), and PyBOP (1.40 g, 2.70 mmol) in DMF (5 ml) was stirred at room temperature for 4 hours and then partitioned in EtOAc (200 ml) and 5% citric acid (100 ml). The organic layer was washed with 5% citric acid (100 ml), 10% K2CO3 (50 ml×2), and water (100 ml), dried (brine, MgSO4) and then concentrated to give an amber oil (1.356 g). An aliquot was analyzed by analytical LC/MS and found to be the desired product, Cbz-Val-Pro-OtBu, along with a minor impurity.

A solution of crude Cbz-Val-Pro-OtBu from the previous step in ethanol (15 ml) and ethyl acetate (50 ml) was charged with 5% Pd on carbon (100 mg) and the mixture stirred at room temperature under hydrogen atmosphere for 20 hours. The reaction was filtered through a pad of celite and the filtrate was concentrated to dryness. The residual oil was coevaporated with ether (100 ml) and then dried under vacuum to provide a white solid (0.76 g). An aliquot was analyzed by analytical LC/MS and found to be the desired product, H-Val-Pro-OtBu, along with the minor impurity from the previous step.

A solution of crude H-Val-Pro-OtBu from the previous step, Cbz-Asp(OtBu)-OSu (840 mg, 2.00 mmol), and DIPEA (870 μl, 2.00 mmol) in DMF (5 ml) was stirred at room temperature for 48 hours and then partitioned in EtOAc (100 ml) and 1M HCl (100 ml). The organic layer was washed with 1M HCl (100 ml), 10% K2CO3 (100 ml, 2 times), and water (100 ml), dried (brine; MgSO4), and concentrated to give a white foam (1.19 g). An aliquot was analyzed by analytical LC/MS and found to be the desired product, Cbz-Asp(OtBu)-Val-Pro-OtBu, along with aminor impurity.

A solution of crude Cbz-Asp(OtBu)-Val-Pro-OtBu from the previous step in ethanol (15 ml) and ethyl acetate (50 ml) was charged with 5% Pd on carbon (100 mg) and the mixture stirred at RT under hydrogen atmosphere for 48 hours. The reaction was filtered through a pad of celite and the filtrate was concentrated to dryness. The residual oil was coevaporated with ether (100 ml) and then dried under vacuum to provide a white solid (0.88 g). An aliquot was analyzed by analytical LC/MS and found to be the desired product, H-Asp(OtBu)-Val-Pro-OtBu.

To a suspension of 4-aminophenylacetic acid (1.64 g, 10.9 mmol) in DMF at room temperature was added o-tolyl isocyanate (1.30 ml, 10.5 mmol) dropwise. The solution was then stirred for 30 minutes before pouring into EtOAc (200 ml) while stirring. The white precipitate was collected and washed with EtOAc (200 ml) and acetonitrile (100 ml) before drying under vacuum resulting in a white powder (1.98 g). An aliquot was analyzed by analytical LC/MS and found to be the desired product, 4-[[[(2-methylphenyl)amino]carbonyl]amino]phenyl-acetic acid (CAP).

To a refluxing mixture of CAP (300 mg, 1.1 mmol) in acetonitrile (5 ml) was added thionyl chloride (85 μl, 1.2 mmol) dropwise. After 15 minutes, HOSu (150 mg, 1.3 mmol) and TEA 350 μl, 2.5 mmol) was added. The reaction became dark brown and was allowed to mix at room temperature for 2 hours before diluting with water (10 ml). The mixture was centrifuged and the supernatant decanted. The solid was washed with water (3×20 ml) and then ether (3×20 ml) before coevaporating with acetonitrile (30 ml) to provide a tan powder (315 mg). An aliquot was analyzed by analytical LC/MS and found to be the desired product, 4-[[[(2-methylphenyl)amino]carbonyl]amino]phenyl-acetate N-hydroxysuccinimide ester (CAP-OSu).

A solution of CAP-OSu (315 mg, 0.83 mmol) and TEA (350 μl, 2.5 mmol) in DMF (5 ml) was treated with H-Lys(Cbz)-OH (280 mg, 1.0 mmol). The mixture was stirred at 60° C. for 1 hour and then diluted with 1 M HCl (25 ml). The precipitate was collected and washed with water (2×20 ml) and ether (20 ml), then coevaporated with ether (20 ml) to give a powder (339 mg). An aliquot was analyzed by analytical LC/MS and found to be the desired product, CAP-Lys(Cbz)-OH.

A solution of CAP-Lys(Cbz)-OH (315 mg, 0.83 mmol) and DIPEA (700 ul, 4.0 mmol) in DMF (5 ml) was added to H-Asp(OtBu)-Val-Pro-OtBu (440 mg, 1.0 mmol) and PyBOP (600 mg, 1.2 mmol). The mixture was stirred at room temperature for 16 hours and then diluted with 5% citric acid (50 ml). The precipitate was collected and washed with 5% citric acid (50 ml), 10% K2CO3 (2×50 ml), and then water (2×50 ml) to give a white powder after coevaporating with methanol (0.79 g). An aliquot was analyzed by analytical LC/MS and found to be the desired product, CAP-Lys(Cbz)-Asp(OtBu)-Val-Pro-OtBu.

A turbid solution of CAP-Lys(Cbz)-Asp(OtBu)-Val-Pro-OtBu (0.79 g, 0.81 mmol) in ethanol (100 ml) and charged with 5% Pd on carbon (100 mg) and the mixture stirred at room temperature under hydrogen atmosphere for 24 hours. The reaction was filtered through a pad of celite and the pad washed with EtOAc/EtOH (1:1,100 ml). The combined filtrate was concentrated to dryness to give an oil (675 mg). An aliquot was analyzed by analytical LC/MS and found to be the desired product, CAP-Lys-Asp(OtBu)-Val-Pro-OtBu.

The following sequence of solid phase chemistry steps were undertaken to prepare the di-t-butyl protected form of SYN00535:

Fmoc-Gly-NovaSynTGT (0.20 mmol/g, 2.00 g) was swelled for 20 minutes in DMF (10 ml). The resin was treated with 20% piperdine in DMF (10 ml) for 10 minutes, 2 times. The resin was washed for 10 minutes with DMF (10 ml), 4 times. The resin was treated with a DIPEA (280 ul; 1.60 mmol, 8 equivalents) and then with a solution of PyBOP (420 mg; 0.80 mmol; 4 equivalents) and N,N-bis[3-(Fmoc-amino)propyl]-glycin sulfate potassium salt (600 mg; 0.80 mmol, 4 eq) in DMF (10 ml) overnight. The resin was washed for 10 minutes with DMF (10 ml), 4 times. The resin was treated with 20% piperdine in DMF (10 ml) for 10 minutes, 2 times. The resin was washed for 10 minutes with DMF (10 ml), 4 times. The resin was treated with a DIPEA (560 ul; 3.2 mmol, 16 eq.) and then with a solution of PyBOP (840 mg; 1.60 mmol; 8 eq.) and N,N-bis[3-(Fmoc-amino)propyl]-glycin sulfate potassium salt (1200 mg; 1.6 mmol, 8 eq) in DMF (10 ml). The mixture was shaken over the weekend. The resin was washed for 10 minutes with DMF (10 ml), 4 times. The resin was treated with 20% piperdine in DMF (10 ml) for 10 minutes, 2 times. The resin was washed for 10 minutes with DMF (10 ml), 4 times.

The resin was dried by washing with DCM (10 ml), 4 hours. A portion of the resin (500 mg, 0.10 mmol) was swelled with DMF (10 ml) for 10 minutes. The resin was treated with a solution of succinic anhydride (200 mg, 2.0 mmol) and DIPEA (350 ul, 2 mmol) in DMF (5 ml) over the weekend. The resin was washed with DMF (10 ml) for 10 min (3 times). The resin was treated with a solution of CAP-Lys-Asp(OtBu)-Val-Pro-OtBu (675 mg, 0.81 mmol), PyBOP (600 mg, 1.2 mmol), and DIPEA (350 ul, 2.0 mmol) in DMF (10 ml) overnight. The resin was filtered and washed with DMF (10 ml) for 10 min (3 times) and then with DCM (10 ml) for 10 min (3 times). The resin was dried by a stream of nitrogen for 3 hours. The resin was treated with 10 ml of cleavage solution (50% ACOH, 40% DCM, 10% MeOH) for 1 h. The resin was filtered off, washed with methanol (20 ml). The filtrate was combined and concentrated. The residue was coevaporated with hexanes (10 ml, 3 times), triturated with ether (10 ml, 2 times), and then dried under vacuum to provide a crude product (96 mg). This crude product (96 mg) was purified in two batches by reverse phase (C18) HPLC (product eluted at 75% acetonitrile) to give after combining and lyophilizing the pure fractions a white solid (32 mg). An aliquot was analyzed by analytical LC/MS and found to be the desired product, the di-t-butyl protected form of SYNO0535.

### Example 5

### Synthesis of SYN00535

The di-t-butyl protected form of SYN00535 from above (9 mg, 2.1 μmol) was treated with TFA (5 ml) for 30 minutes and then concentrated by a stream of nitrogen gas. The residue was dissolved in water (15 ml) with a minimum amount of acetonitrile and then lyophilized to give a fluffy white powder that was triturated with ether (8 mg). An aliquot was analyzed by analytical LC/MS and found to be the desired product, SYN00535.

### Example 6

### Synthesis of SYN00534

A solution of the di-t-butyl protected form of SYNO0535 from above (21 mg, 4.5 μmol), HCl/H-Gly-SBn (10 mg, 45 μmol), and HBTU (20 mg, 50 μmol) in DMF (500 μl) and DIPEA (15 μl, 86 μmol)) was stirred in a vial for 2 hours and then diluted with 1:1 water/acetonitrile (with 0.1% TFA). The clear solution was loaded onto a reverse phase (Cl8) semiprep HPLC and eluted with a water/acetonitrile gradient. The pure fractions (eluting at 77% acetonitrile) were combined and lyophilized to give a white powder. This material was treated with TFA (2 ml) for 30 minutes before concentrating by a stream of nitrogen gas. The residue was triturated with ether (3×10 ml) to provide a white solid (13 mg). An aliquot was analyzed by analytical LC/MS and found to be the desired product, SYN00534.

### Example 7

### Synthesis of SYN00534-Fc

CysFc (1.0 mg, 1 mg/ml final concentration) and SYN00534 (1.3 mg, approximately 10 molar equivalents) were incubated for 18 hours at room temperature in 50 mM Tris 8 and 50 mM MESNA. The solution was then loaded into a dialysis cassette (Pierce Slide-A-Lyzer) (Pierce, Rockford, Ill.) and dialyzed with 1000 ml of PBS 5 times (1 hour, 2 hours, 18 hours, 3 hours, and then 20 hours). Analysis by SDS-PAGE (Tris-Gly gel) using reducing sample buffer indicated the presence of a new band approximately 4 kDa larger than the Fc control (approx. 60% conversion to the conjugate). Previous N-terminal sequencing of Cys-Fc and unreacted Cys-Fc indicated that the signal peptide is incorrectly processed in a fraction of the molecules, leaving a mixture of (Cys)-Fc, which will react through native ligation with peptide-thioesters, and (Val)-(Gly)-(Cys)-Fc, which will not. As the reaction conditions are insufficient to disrupt the dimerization of the CysFc molecules, this reaction generated a mixture of SYN00534-Fc:SYN00534-Fc homodimers, SYN00534-Fc: Fc heterodimers, and CysFc:CysFc homodimers.

### Example 8

### Peptide-dendrimer-Fc coniugates

For N-linked peptides: The dendrimeric resin prepared up to and including Step 15 of the procedure described above can be utilized for the synthesis of Peptide-dendrimer-Fc's. Instead of utilizing CAP-Lys-Asp(OtBu)-Val-Pro-OtBu in Step 16, a peptide with a free amine and appropriately protected with TFA labile protecting groups can be used. This material could then be carried forward as described in steps 17,18, and 19, as was described for the synthesis of SYN00534, and then as described for SYN00534-Fc.

For C-linked peptides the dendrimeric resin prepared up to and including Step 13 of the procedure described above can be utilized for the synthesis of Peptide-dendrimer-Fc's. Steps 14 and 15 could be skipped and instead of utilizing CAP-Lys-Asp(OtBu)-Val-Pro-OtBu in Step 16, a peptide with a free carboxyl group and appropriately protected with TFA labile protecting groups can be used. This material could then be carried forward as described in steps 17,18, and 19, as described for the synthesis of SYN00534, and then as described for SYN00534-Fc.

All references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

1. A method of treating a subject having a disease or condition, said method comprising
administering a chimeric protein to said subject such that the disease or condition is treated,
wherein said chimeric protein comprises a biologically active molecule having a modification and
wherein, said modification comprises linking said biologically active molecule to at least a portion of an immunoglobulin constant region such that said biologically active molecule having the modification binds less serum albumin than the same biologically active molecule without said modification.

2. The method of claim 1, wherein the at least a portion of an immunoglobulin constant region comprises the Fc fragment of an immunoglobulin.

3. The method of claim 2, wherein said Fc fragment of an immunoglobulin is an FcRn binding partner.

4. The method of claim 3, wherein the FcRn binding partner is a peptide mimetic of an Fc fragment of an immunoglobulin.

5. The method of claim 1 or 3, wherein said biologically active molecule is a protein.

6. The method of claim 1 or 3, wherein said biologically active molecule is a peptide.

7. The method of claim 1 or 3, wherein said biologically active molecule is a nucleic acid.

8. The method of claim 7, wherein said nucleic acid is an DNA molecule or an RNA molecule.

9. The method of claim 1 or 3, wherein the biologically active molecule is a growth factor or hormone, or an analog thereof.

10. The method of claim 9, wherein the biologically active molecule is GnRH.

11. The method of claim 6, wherein the biologically active molecule is leuprolide.

12. The method of claim 1 or 3, wherein said biologically active molecule is a small molecule.

13. The method of claim 12, wherein said small molecule is a VLA4-antagonist.

14. The method of claim 1 or 3, wherein the serum albumin is human serum albumin.

15. A method of increasing the unbound serum concentration of a biologically active molecule, said method comprising
administering a chimeric protein comprising a biologically active molecule, said biologically active molecule having a modification,
wherein said modification comprises linking said biologically active molecule to at least a portion of an immunoglobulin constant region such that said biologically active molecule having said modification binds less serum albumin compared to the same biologically active molecule without said modification, thereby increasing the unbound serum concentration of said biologically active molecule.

16. The method of claim 15, wherein said at least a portion of an immunoglobulin constant region comprises the Fc fragment of an immunoglobulin.

17. The method of claim 16, wherein said Fc fragment of an immunoglobulin is an FcRn binding partner.

18. The method of claim 17, wherein the FcRn binding partner is a peptide mimetic of an Fc fragment of an immunoglobulin.

19. The method of claim 15 or 17, wherein said biologically active molecule is a protein.

20. The method of claim 15 or 17, wherein said biologically active molecule is a peptide.

21. The method of claim 15 or 17, wherein said biologically active molecule is a growth factor or hormone.

22. The method of claim 21, wherein the growth factor or hormone is GnRH.

23. The method of claim 15 or 17, wherein said biologically active molecule is a nucleic acid.

24. The method of claim 23, wherein said nucleic acid is an DNA molecule or an RNA molecule.

25. The method of claim 15 or 17, wherein said biologically active molecule is a small molecule.

26. The method of claim 15 or 17, wherein said small molecule is a VLA4-antagonist.

27. The method of claim 15 or 17, wherein the subject is human.

28. The method of claim 15 or 17, wherein the biologically active molecule is a growth factor or hormone or analog thereof.

29. The method of claim 28, wherein the growth factor or hormone analog is leuprolide.

30. The method of claim 28, wherein the growth factor or hormone is GnRH.

31. The method of claim 15 or 17, wherein the serum albumin is human serum albumin.

32. A chimeric protein comprising a biologically active molecule having a modification, wherein said modification comprises linking said biologically active molecule to at least a portion of an immunoglobulin constant region, such that said biologically active molecule binds substantially no serum albumin compared to the same biologically active molecule without said modification.

33. The chimeric protein of claim 32, wherein said at least a portion of an immunoglobulin constant region comprises the Fc fragment of an immunoglobulin.

34. The chimeric protein of claim 33, wherein said Fc fragment of an immunoglobulin is an FcRn binding partner.

35. The method of claim 34, wherein the FcRn binding partner is a peptide mimetic of an Fc fragment of an immunoglobulin.

36. The chimeric protein of claim 32 or 34, wherein said biologically active molecule is a protein.

37. The chimeric protein of claim 32 or 34, wherein said biologically active molecule is a peptide.

38. The chimeric protein of claim 32 or 34, wherein said biologically active molecule is a growth factor or hormone.

39. The chimeric protein of claim 38, wherein the growth factor or hormone is GnRH.

40. The chimeric protein of claim 32 or 34, wherein said biologically active molecule is a nucleic acid.

41. The chimeric protein of claim 40, wherein said nucleic acid is an DNA molecule or an RNA molecule.

42. The chimeric protein of claim 32 or 34, wherein said biologically active molecule is a small molecule.

43. The method of claim 42, wherein said small molecule is a VLA4-antagonist.

44. The chimeric protein of claim 32 or 34, wherein the serum albumin is human serum albumin.

45. A chimeric protein comprising a biologically active molecule having a modification, wherein said modification comprises linking said biologically active molecule to at least a portion of an immunoglobulin constant region, such that said biologically active molecule binds less serum albumin compared to the same biologically active molecule without said modification.

46. The chimeric protein of claim 45, wherein said portion of an immunoglobulin constant region comprises the Fc fragment of an immunoglobulin.

47. The chimeric protein of claim 45, wherein said portion of an immunoglobulin constant region of an immunoglobulin is an FcRn binding partner.

48. The method of claim 47, wherein the FcRn binding partner is a peptide mimetic of an Fc fragment of an immunoglobulin.

49. The chimeric protein of claim 45 or 47, wherein said biologically active molecule is a protein.

50. The chimeric protein of claim 45 or 47, wherein said biologically active molecule is a peptide.

51. The chimeric protein of claim 45 or 47, wherein said biologically active molecule is a nucleic acid.

52. The chimeric protein of claim 51, wherein said nucleic acid is an DNA molecule or an RNA molecule.

53. The chimeric protein of claim 45 or 47, wherein the biologically active molecule is a growth factor or hormone, or an analog thereof.

54. The chimeric protein of claim 53, wherein the growth factor or hormone analog is leuprolide.

55. The chimeric protein of claim 53, wherein the growth factor or hormone is GnRH.

56. The chimeric protein of claim 45 or 47, wherein said biologically active molecule is a small molecule.

57. The chimeric protein of claim 56, wherein said small molecule is a VLA4-antagonist.

58. The chimeric protein of claim 45 or 47, wherein the serum albumin is human serum albumin.

59. A kit for detecting serum albumin binding to a biologically active molecule comprising a biologically active molecule fused to at least a portion of an immunoglobulin and a container.

60. The kit of claim 59, wherein said at least a portion of an immunoglobulin constant region comprises the Fc fragment of an immunoglobulin.

61. The kit of claim 59, wherein the portion of the immunoglobulin is an FcRn binding partner.

62. The chimeric protein of claim 57, wherein said chimeric protein comprises a dendrimeric linker.

63. A method of making a chimeric protein comprising a biologically active molecule having a modification, wherein said modification comprises linking said biologically active molecule to at least a portion of an immunoglobulin constant region, such that said biologically active molecule binds less serum albumin compared to the same biologically active molecule without said modification said method comprising
a) recombinantly expressing at least a portion of an immunoglobulin constant region;
b) chemically synthesizing, or recombinantly expressing a biologically active molecule comprising at least one linker; and
c) combining the portion of an immunoglobulin constant region of a) with the biologically active molecule of b) to make a chimeric protein.

64. The method of claim 63, wherein the linker is a dendrimer.

65. The method of claim 64, wherein the biologically active molecule is a VLA4 antagonist.


SEQUENCE LISTING (21 sequences)

SEQ ID NO: 1 - protein, 36 aa, Human immunodeficiency virus
YTSLIHSLIEESQNQQEKNEQELLELDKWASLWNWF

SEQ ID NO: 2 - protein, 37 aa, Human immunodeficiency virus
NNLRAIEAQQHLLQLTVWGIKQLQARILAVERYLKDQ

SEQ ID NO: 3 - protein, 39 aa, Human immunodeficiency virus
WQEWEQKITALLEQAQIQQEKNEYELQKLDKWASLWEWF

SEQ ID NO: 4 - protein, 102 aa, Human immunodeficiency virus
SGIVQQQNNLLRAIEAQQHLLQLTVWGIKQCCGRISGIVQQQNNLLRAIEQQHLLQLTVWGIKQLQARSGGRGGWMEWDREINNYTSLIHSLIEESQNQQEK

SEQ ID NO: 5 - protein, 213 aa, Human immunodeficiency virus
QLLSGIVQQQNNLRAIEAQQHLLQLTVWGIKQLQARILAGGSGGHTTWMEWDREINNYTSLIHSLIEESQNQQEKNEQELLEGSSGGQLLSGIVQQQNNLRAIEAQQHLLQLTVWGIKQLQARILAGGSGGHTTWMEWDREINNYTSLIHSLIEESQNQQEKNEQELLEGSSGGQLLSGIVQQQNNLRAIEAQQHLLQLTVWGIKQLQARILA

SEQ ID NO: 6 - DNA, 687 bp, Homo sapiens
gaaccaaagagctccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctcgctgagc

SEQ ID NO: 7 - protein, 228 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNFSCSVMHEALHNHYTQKSLSLS

SEQ ID NO: 8 - protein, 11 aa, Artificial Sequence
  Description of Artificial Sequence Synthetic peptide
PKNSSMISNTP

SEQ ID NO: 9 - protein, 7 aa, Artificial Sequence
  Description of Artificial Sequence Synthetic peptide
HQSLGTQ

SEQ ID NO: 10 - protein, 8 aa, Artificial Sequence
  Description of Artificial Sequence Synthetic peptide
HQNLSDGK

SEQ ID NO: 11 - protein, 8 aa, Artificial Sequence
  Description of Artificial Sequence Synthetic peptide
HQNISDGK

SEQ ID NO: 12 - protein, 8 aa, Artificial Sequence
  Description of Artificial Sequence Synthetic peptide
VISSHLGQ

SEQ ID NO: 13 - protein, 30 aa, Artificial Sequence
  Description of Artificial Sequence Linker peptide
GGSGGSGGSGGSGGSGGSGGSGGSGGSGGS

SEQ ID NO: 14 - protein, 3 aa, Artificial Sequence
  Description of Artificial Sequence Linker peptide
GGG

SEQ ID NO: 15 - protein, 7 aa, Artificial Sequence
  Description of Artificial Sequence Linker peptide
SGGSGGS

SEQ ID NO: 16 - protein, 15 aa, Artificial Sequence
  Description of Artificial Sequence Linker peptide
GGSGGSGGSGGSGGG

SEQ ID NO: 17 - protein, 18 aa, Artificial Sequence
  Description of Artificial Sequence Linker peptide
GGSGGSGGSGGSGGSGGS

SEQ ID NO: 18 - protein, 4 aa, Homo sapiens
ELLG

SEQ ID NO: 19 - protein, 10 aa, Artificial Sequence
  Description of Artificial Sequence Linker peptide
GGGGGGGGGG

SEQ ID NO: 20 - protein, 4 aa, Artificial Sequence
  Description of Artificial Sequence Synthetic peptide
KDVP

SEQ ID NO: 21 - protein, 4 aa, Artificial Sequence
  Description of Artificial Sequence Synthetic peptide
KDVP